(19) United States
(12) Patent Application Publication
(10) Pub. No.: US 2023/0115516 A1
(43) Pub. Date: Apr. 13, 2023

(54) CULTURE APPARATUS AND CULTURE METHOD

(71) Applicant: HONDA MOTOR CO., LTD., Minato-ku, Tokyo (JP)

(72) Inventors: Shohei Kinoshita, Haga-gun, Tochigi-ken (JP); Nozomi Shiobara, Haga-gun, Tochigi-ken (JP); Kenji Machida, Haga-gun, Tochigi-ken (JP); Minoru Goto, Haga-gun, Tochigi-ken (JP); Mizuho Doi, Haga-gun, Tochigi-ken (JP)

(21) Appl. No.: 17/913,857

(22) PCT Filed: Mar. 24, 2021
(86) PCT No.: PCT/JP2021/012190

§ 371 Date: Sep. 23, 2022

(30) Foreign Application Priority Data
Mar. 30, 2020 (JP) 2020-059330

(51) Int. Cl. C12M 1/00; A01G 33/00

(52) U.S. Cl. CPC C12M 21/02; A01G 33/00; C12M 29/24

(57) ABSTRACT

Provided are a culture apparatus and a culture method with which microalgae can be cultured satisfactorily. The culture apparatus includes a plurality of culture tanks and a water storage tank, and cultures microalgae in a culture solution. Each of the plurality of culture tanks has a translucent accommodating portion containing the culture solution and microalgae. The volumes of the accommodating portions of the plurality of culture tanks are different from each other. The water storage tank has a translucent water storage unit for storing a stored water. The plurality of culture tanks are selectively arranged in the water storage unit.

### TECHNICAL FIELD

The present invention relates to a culture device (culture apparatus) and a culture method for culturing microalgae within a culture solution.

### BACKGROUND ART

For example, as shown in JP 4523187 B2, a culture device for culturing microalgae within a culture solution is known. Such a culture device is equipped with a plurality of culturing tanks disposed at a predetermined interval, and an interval adjustment unit which is capable of adjusting an interval between the culturing tanks. By providing the interval adjustment unit in this manner, the interval between the culturing tanks can be adjusted in accordance with the altitude of the sun or the like, in order to improve the culturing efficiency of the microalgae, for example, by causing a light-receiving area of the culturing tanks to be increased regardless of the altitude of the sun in each of the seasons.

### SUMMARY OF THE INVENTION

Incidentally, in this type of culture device, the temperature or the like of the culture solution changes, for example, in accordance with seasonal changes in the outdoor environment of the installation location in which the device is installed. Therefore, as discussed above, even if the interval between the culturing tanks is capable of being adjusted, a concern arises in that it is difficult to maintain the culture solution at a temperature suitable for culturing the microalgae, and as a result, the microalgae cannot be cultured satisfactorily.

The present invention has been devised in order to solve the aforementioned problems, and has the object of providing a culture device and a culture method which are capable of culturing microalgae in a satisfactory manner.

One aspect of the present invention is characterized by a culture device that cultures microalgae in a culture solution, the culture device including: a plurality of culturing tanks each including an accommodation unit that transmits light and is configured to accommodate the culture solution and the microalgae, wherein capacities of the accommodation units differ from each other; and a water storage tank including a water storage unit which transmits light and in which stored water is stored, wherein the plurality of culturing tanks are selectively disposed inside the water storage unit.

Another aspect of the present invention is characterized by a culture method using a culture device that cultures microalgae in a culture solution, wherein the culture device includes a culturing tank including an accommodation unit that transmits light and is configured to accommodate the culture solution and the microalgae as contents, and a water storage tank including a water storage unit which transmits light and in which stored water is stored, the culturing tank being allowed to be installed inside the water storage unit, the culture method comprising a determination step of determining an outdoor environment of an installation location in which the culture device is installed, and a volume occupancy rate adjustment step of setting, based on a determination result of the determination step, a volume occupancy rate which is a ratio of a content volume of the contents accommodated in the accommodation unit inside the water storage unit, to a total volume of the content volume and a stored water volume of the stored water.

In the culture device equipped with the plurality of culturing tanks in which the capacities of the accommodation units differ from each other, it becomes possible to selectively dispose the plurality of culturing tanks in the water storage unit. Consequently, it is possible to adjust the volume occupancy rate, which is a ratio of the content volume in the accommodation unit disposed in the water storage unit, to the total volume of the stored water volume and the content volume.

In the case that the volume occupancy rate is decreased, and the ratio of the stored water volume to the total volume is increased, for example, it becomes possible to effectively obtain a cooling action of the stored water with respect to the culturing tanks. For this reason, for example, even in the case that the outside air temperature of the outdoor environment of the installation location of the culture device is higher than a predetermined temperature suitable for culturing in the culturing tank, a rise in temperature inside the accommodation unit can be suppressed due to the cooling action of the stored water.

On the other hand, the culture solution in which the microalgae is contained is colored, such as a green color, and absorbs infrared rays and the temperature thereof rises more easily than the stored water which is transparent. Therefore, in the case that the volume occupancy rate is increased, and the ratio of the content volume to the total volume is increased, for example, it becomes possible to effectively obtain a heat retaining action of the culturing tank. For this reason, for example, even in the case that the outside air temperature of the outdoor environment of the installation location of the culture device is lower than the predetermined temperature suitable for culturing in the culturing tank, lowering of the temperature inside the accommodation unit can be suppressed.

From the above, according to the present invention, by enabling the volume occupancy rate to be adjusted, for example, it becomes easy to maintain the culture solution at a temperature suitable for culturing the microalgae, and it becomes possible to culture the microalgae in a satisfactory manner.

### BRIEF DESCRIPTION OF DRAWINGS

FIG. 1 is a schematic cross-sectional view of a first culturing tank of a culture device according to an embodiment of the present invention;

FIG. 2 is a cross-sectional view taken along line II-II of FIG. 1;

FIG. 3 is a schematic cross-sectional view of a second culturing tank of the culture device according to the embodiment of the present invention;

FIG. 4 is a cross-sectional view taken along line IV-IV of FIG. 3;

FIG. 5 is a schematic perspective view of a water storage tank of the culture device according to the embodiment of the present invention;

FIG. 6A is a schematic plan view for describing the culture device in which the first culturing tank is installed in a water storage unit of the water storage tank;

FIG. 6B is a schematic front view of the culture device shown in FIG. 6A;

FIG. 7A is a schematic plan view for describing the culture device in which the second culturing tank is installed in the water storage unit of the water storage tank; and

FIG. 7B is a schematic front view of the culture device shown in FIG. 7A.

### DESCRIPTION OF THE INVENTION

A preferred embodiment of a culture device and a culture method according to the present invention will be presented and described in detail below with reference to the accompanying drawings. It should be noted that, in the drawings referred to hereinafter, constituent elements that exhibit the same or similar functions and effects may be designated by the same reference numerals, and repeated description thereof may be omitted.

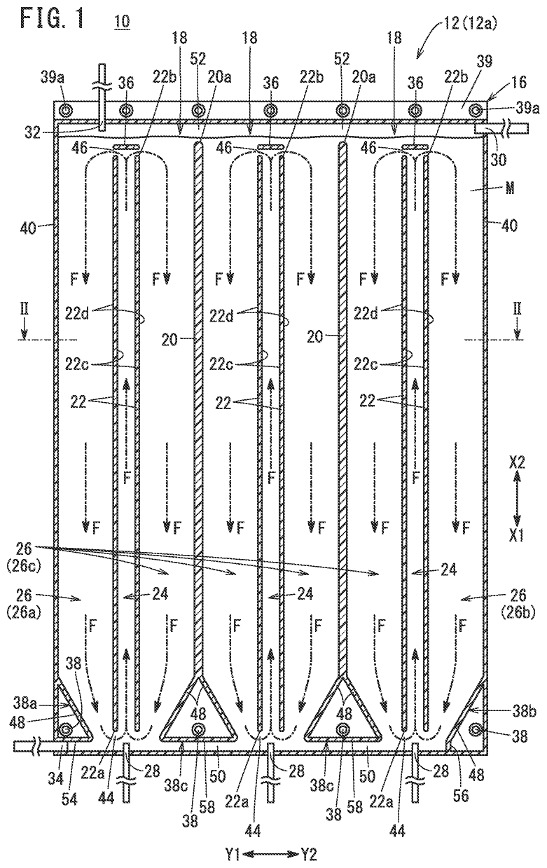

A culture device 10 according to the present embodiment shown in FIGS. 1 to 5 cultures microalgae by supplying light and a gas such as a carbon dioxide gas or a gas containing carbon dioxide to microalgae (not shown) existing within a culture solution that contains water, so that the microalgae proliferate while carrying out photosynthesis. The microalgae that are capable of being cultured by the culture device 10 are not particularly limited. However, in the case of using the cultured microalgae to produce, for example, a biofuel such as ethanol, it is preferable to culture microalgae which are classified as Chlorophyceae (for example, Chlamydomonas, chlorella), Prasinophytes, Cryptophytes, and Cyanobacteria (e.g., Spirulina). In addition to water, the culture solution may contain nutrients or the like necessary for culturing the microalgae. The gas preferably contains a carbon dioxide gas discharged from a factory or the like.

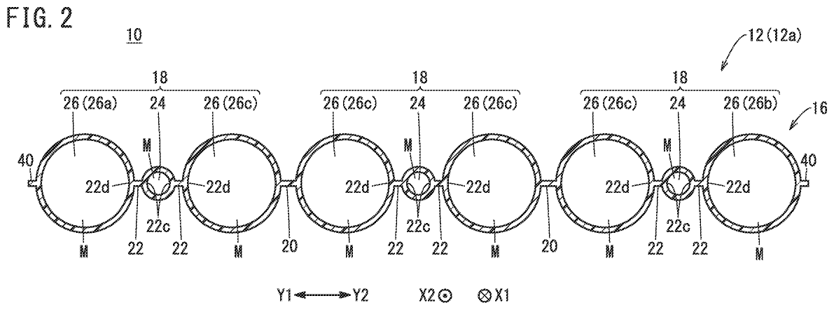

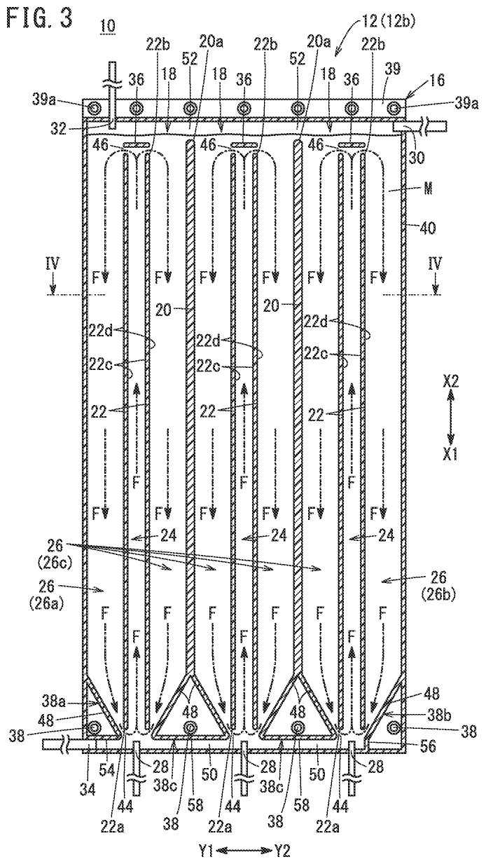

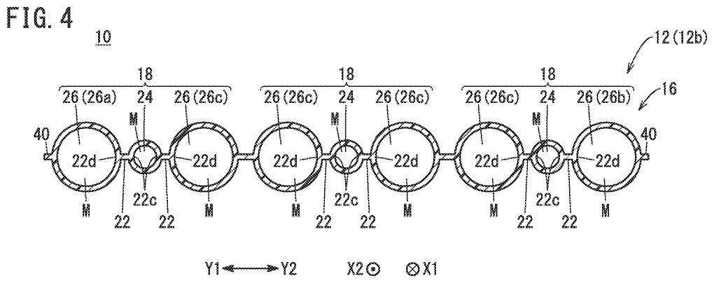

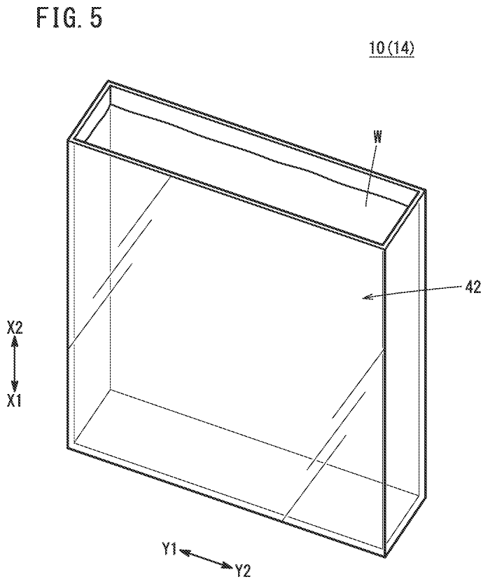

As an environment that can be irradiated with light having a wavelength (for example, 400 to 700 nm) required for growth of the microalgae, for example, the culture device 10 is installed outdoors where it can be irradiated with sunlight. Further, the culture device 10 also includes a plurality of culturing tanks 12 as shown in FIGS. 1 to 4, and a water storage tank 14 as shown in FIG. 5. According to the present embodiment, the plurality of culturing tanks 12 are made up from two tanks, including a first culturing tank 12a as shown in FIG. 1 and FIG. 2, and a second culturing tank 12b as shown in FIG. 3 and FIG. 4, but the number of the culturing tanks 12 is not necessarily limited to two. Moreover, hereinafter, in the case that the first culturing tank 12a and the second culturing tank 12b are not distinguished in particular, the culturing tanks may also be collectively referred to as “culturing tanks 12”.

As shown in FIG. 1 and FIG. 2, the first culturing tank 12a is equipped with a main body portion 16 formed from a material that is flexible and transmits light, such as linear low density polyethylene (LLDPE). In this instance, the term “material that transmits light” implies that light having a wavelength required for growth of the microalgae can be transmitted through such a material. According to the present embodiment, the main body portion 16 is formed in a hollow shape (bag shape) by superimposing two sheets made of the aforementioned material; however the present invention is not particularly limited to pthis feature.

As shown in FIGS. 1 and 2, the main body portion 16 of the first culturing tank 12a includes accommodation units 18, partitioning members 20, joint members 22, guide units 24, circulation units 26, gas supply ports 28, a culture solution supply port 30, a gas discharge port 32, a collection port 34, facing portions 36, and sealing members 38a, 38b, and 38c.

The accommodation units 18 are provided in plurality (three in the present embodiment) in the main body portion 16, by the interior of the hollow main body portion 16 being partitioned by the partitioning members 20. The culture solution and the microalgae are accommodated as contents M in each of the accommodation units 18. A gas is supplied to each of the accommodation units 18 through the gas supply ports 28, which are connected to a non-illustrated gas supply mechanism.

The plurality of accommodation units 18 are integrally surrounded by enclosures 40 formed by joining together inner wall surfaces of the main body portion 16 along the outer peripheral edge part of the main body portion 16 by welding or the like. In other words, the culture device 10 is a so-called closed system, in which the microalgae are cultured within the culture solution that is accommodated in the interior of the sealed accommodation units 18.

Further, as will be described later, when the main body portion 16 is installed in a water storage unit 42 of the water storage tank 14 shown in FIG. 5, gas is supplied to the interior of the accommodation units 18 from a lower side (the side of the arrow X1) toward an upper side (the side of the arrow X2) in the vertical direction. The main body portion 16 and the accommodation units 18, respectively, are formed in a rectangular shape, with the direction in which the gas is supplied (the vertical direction) being the long side, and the horizontal direction being the short side when viewed from the front.

The joint members 22 are formed in the interior of each of the accommodation units 18 by joining together the inner wall surfaces of the main body portion 16 by welding or the like, and extend in the direction (the vertical direction, the direction of the arrows X1 and X2) in which the gas is supplied. The length of the joint members 22 in the direction (the vertical direction) in which the joint members extend is set to be shorter than the length of the accommodation units 18 in the vertical direction. One end parts 22a and other end parts 22b of the joint members 22 in the direction in which the joint members extend are formed respectively in an arcuate shape.

A pair of the joint members 22 which face toward each other with an interval therebetween are formed respectively on each of the accommodation units 18. The guide unit 24 is provided between inner side surfaces 22c of the pair of joint members 22, and moreover, the circulation units 26 are formed respectively on the sides of outer side surfaces 22d of the pair of joint members 22. In other words, the guide unit 24 and the circulation units 26 lie adjacent to each other with the joint members 22 being interposed therebetween, and are disposed along the direction in which the joint members 22 extend, in the interior of each of the accommodation units 18. Moreover, only one joint member 22 may be provided in each of the accommodation units 18, and one guide unit 24 may be disposed on one side and one circulation unit 26 may be disposed on another side of the joint member 22 in the direction of the arrows Y1 and Y2.

The guide unit 24 is provided in substantially the center of each of the accommodation units 18 in a short-side direction thereof (the direction of the arrows Y1 and Y2) so as to extend in a long-side direction thereof (the direction of the arrows X1 and X2). The circulation units 26 are provided on both sides of the guide unit 24 in the short-side direction of the accommodation units 18 so as to extend in the long-side direction thereof. As shown in FIG. 2, each of the guide units 24 and the circulation units 26 in which the contents M are accommodated are formed in a substantially cylindrical shape when viewed in the vertical direction.

As shown in FIG. 1, the guide units 24 and the circulation units 26 communicate with each other through guide unit inlet ports 44 provided on an upstream side (the lower side, the side of the arrow X1) in the direction in which the gas is supplied, and guide unit outlet ports 46 provided on a downstream side (the upper side, the side of the arrow X2) in the direction in which the gas is supplied. It should be noted that, hereinafter, the upstream side in the direction in which the gas is supplied is also simply referred to as an “upstream side”, and the downstream side in the direction in which the gas is supplied is also simply referred to as a “downstream side”. In the present embodiment, the upstream side is a lower side in the vertical direction. Further, the downstream side is an upper side in the vertical direction.

In accordance with the foregoing, by the length of the joint members 22 in the direction of extension thereof being shorter than the length of the accommodation units 18 in the vertical direction, the guide unit inlet ports 44 are disposed between the one end parts 22a (the end parts on the side of the arrow X1) of the joint members 22 in the direction of extension thereof and the enclosures 40. Further, the guide unit outlet ports 46 are disposed between the other end parts 22b (the end parts on the side of the arrow X2) of the joint members 22 in the direction of extension thereof and the enclosures 40.

A pair of inclined sections 48 are formed in the circulation units 26 on the side of the guide unit inlet ports 44 (the side of the arrow X1), by joining together the inner wall surfaces of the main body portion 16 by welding or the like. The respective inclined sections 48 are inclined in a direction away from the guide units 24 from the lower side (the side of the arrow X1) toward the upper side (the side of the arrow X2) when the main body portion 16 is installed in the water storage unit 42. According to the present embodiment, the lower end parts of the inclined sections 48 are arranged below the one end parts 22a of the joint members 22. It should be noted that the inclined sections 48 need not necessarily be provided in the main body portion 16.

The gas supply ports 28 are disposed respectively underneath the guide units 24 of the accommodation units 18, and enable the gas from the gas supply mechanism to be supplied to the guide units 24. By supplying the gas via the gas supply ports 28 in this manner, the gas can be made to flow through the guide units 24 in the direction (the vertical direction) in which the gas is supplied. In this manner, when the gas flows through the guide units 24, a culture solution flow F is generated in which the culture solution (the contents M) inside the circulation units 26 flows into the guide units 24 from the guide unit inlet ports 44, and the culture solution inside the guide units 24 flows out from the guide unit outlet ports 46 into the circulation units 26.

The culture solution supply port 30 is disposed, for example, on an upper end side of the main body portion 16. Further, the culture solution supply port 30 is connected to a non-illustrated culture solution supply mechanism, and enables the culture solution to be supplied to the interior of the accommodation units 18. Moreover, the microalgae may be supplied to the interior of the accommodation units 18 together with the culture solution via the culture solution supply port 30.

The gas discharge port 32 is disposed on the upper end side of the main body portion 16, and enables the gas in the interior of the accommodation units 18 to be discharged. As the gas in the interior of the accommodation units 18, there may be cited a residual gas that has not been consumed by photosynthesis of the microalgae, oxygen gas generated by photosynthesis, and the like, from among the gases supplied from the gas supply ports 28.

The collection port 34 is disposed, for example, on a lower end side of the main body portion 16, and enables the contents M in the accommodation units 18 to be collected. The culture solution supply port 30 and the collection port 34 are provided so as to be capable of opening and closing, and can be placed in a closed state except when the culture solution is supplied to and discharged from the accommodation units 18.

The partitioning members 20 are formed by joining together the inner wall surfaces of the main body portion 16 by welding or the like, and extend in the direction in which the gas is supplied. The length of the partitioning members 20 in the direction of extension thereof is set to be shorter than the length of the accommodation units 18 in the vertical direction, and longer than the length of the joint members 22 in the direction of extension thereof. End parts 20a of the partitioning members 20 on the side of the arrow X2 in the direction of extension thereof are formed in an arcuate shape. The end parts 20a of the partitioning members 20 on the side of the arrow X2 are arranged on the downstream side of the other end parts 22b of the joint members 22 (on the upper side, the side of the arrow X2). The inclined sections 48, which branch off from the partitioning members 20 and extend toward the guide unit inlet ports 44 of the accommodation units 18, are integrally provided on the respective partitioning members 20 on the side of the arrow X1 in the direction of extension thereof.

On both sides of the partitioning members 20 of the main body portion 16 in the direction of the arrows Y1 and Y2, the accommodation units 18 are disposed respectively along the direction in which the partitioning members 20 extend (in the direction of the arrows X1 and X2). According to the present embodiment, the main body portion 16 is provided with two partitioning members 20 at an interval in the direction of the arrows Y1 and Y2. Therefore, the main body portion 16 includes three accommodation units 18, which lie adjacent to each other with the partitioning members 20 being interposed therebetween. Each of the accommodation units 18 includes one guide unit 24, and two circulation units 26 provided on both sides of the guide unit 24. In other words, the main body portion 16 has a total of three guide units 24, and six circulation units 26.

As shown in FIG. 2, in the main body portion 16, when viewed in the vertical direction, the inner diameters of the guide units 24 are substantially the same, the inner diameters of the circulation units 26 are substantially the same, and the inner diameters of the guide units 24 are smaller than the inner diameters of the circulation units 26. However, the relationship between the size of the inner diameters of the guide units 24 and the size of the inner diameters of the circulation units 26 is not particularly limited.

Further, the partitioning members 20 need not necessarily be provided in the main body portion 16. In this case, the main body portion 16 has one accommodation unit 18. Furthermore, the number of the partitioning members 20 provided in the main body portion 16 is not necessarily limited to two. For example, by providing only one partitioning member 20, the main body portion 16 may include two accommodation units 18, and by providing three or more partitioning members 20, the main body portion 16 may include four or more accommodation units 18. Further, in the case that the main body portion 16 includes a plurality of the accommodation units 18, instead of the direction (the vertical direction) in which the gas is supplied, a direction (the horizontal direction) perpendicular to the direction in which the gas is supplied may be set as the long-side direction of the main body portion 16.

The accommodation units 18 communicate with each other via upstream side communication ports 50 provided on the upstream side (the side of the arrow X1) and downstream side communication ports 52 provided on the downstream side (the side of the arrow X2) of the accommodation units 18. According to the present embodiment, as noted previously, the upstream side communication ports 50 are disposed between the inclined sections 48 that are integrally provided at the ends of the partitioning members 20 on the side of the arrow X1 in the direction of extension thereof, and the enclosures 40. Further, the downstream side communication ports 52 are disposed between the end parts 20a of the respective partitioning members 20 on the side of the arrow X2, and the enclosures 40. The other end parts 22b of the joint members 22 are arranged on the upstream side of the downstream side communication ports 52 (on the lower side, the side of the arrow X1).

The facing portions 36 are disposed respectively on the downstream side of the other end parts 22b of the joint members 22 (on the upper side, the side of the arrow X2) and on the upstream side of the downstream side communication ports 52 (on the lower side, the side of the arrow X1), in a manner so as to face toward the guide units 24 of the accommodation units 18. The facing portions 36 are formed by joining together the inner wall surfaces of the main body portion 16 by welding or the like, and extend along a direction (in the present embodiment, the direction of the arrows Y1 and Y2) intersecting the direction in which the gas is supplied. Both end parts of the facing portions 36 in the direction of extension thereof are preferably formed in an arcuate shape. It should be noted that the facing portions 36 need not necessarily be provided in the main body portion 16.

The sealing members 38a, 38b, and 38c are isolated from the accommodation units 18, by being formed by joining together the inner wall surfaces of the main body portion 16. In other words, inward flowing of the contents M into the inner sides of the sealing members 38a, 38b, and 38c is restricted. From among the plurality of circulation units 26 that are provided inside the accommodation units 18 in the manner described above, in the circulation unit 26 (a circulation unit 26a) that is disposed on the end on the side of the arrow Y1 closest to the collection port 34, a triangular sealing member 38a is formed by the inclined section 48, a bottom side portion 54, and a portion of the enclosure 40. The bottom side portion 54 extends along the short-side direction of the accommodation units 18 from a lower end part of the inclined section 48 toward a side (the side of the arrow Y1) away from the guide unit 24.

From among the plurality of circulation units 26 inside the accommodation units 18, in the circulation unit 26 (a circulation unit 26b) that is disposed on the end on the side of the arrow Y2 farthest away from the collection port 34, a quadrangular sealing member 38b is provided. The sealing member 38b is formed by the inclined section 48, an extending portion 56 that extends downward from the lower end part of the inclined section 48, and a corner portion of the enclosure 40.

From among the plurality of circulation units 26 inside the accommodation units 18, in the circulation units 26 (circulation units 26c), which lie adjacent to each other via the partitioning member 20, triangular sealing members 38c are formed by integrally providing, between the lower ends of the inclined sections 48 that branch off from the partitioning members 20, bottom side portions 58 that extend along the short-side direction.

It should be noted that the sealing members 38a, 38b, and 38c are not necessarily limited to being formed in the quadrangular shape or the triangular shape described above, as long as they are formed in a manner so as to be isolated from the accommodation units 18 by joining together the inner wall surfaces of the main body portion 16 by welding or the like.

Position fixing holes 38 are provided respectively in the sealing members 38a, 38b, and 38c. The position fixing holes 38 enable non-illustrated support members or the like to be inserted therethrough when the main body portion 16 is installed in the water storage unit 42. It should be noted that the sealing members 38a, 38b, and 38c and the position fixing holes 38 need not necessarily be provided in the main body portion 16.

Above the accommodation units 18 in the main body portion 16, a fixing member 39 is provided that is isolated from the interior of the accommodation units 18. Similar to the position fixing holes 38, through holes 39a through which support members or the like are inserted when the main body portion 16 is installed in the water storage unit 42 are provided in the fixing member 39.

The second culturing tank 12b shown in FIGS. 3 and 4 is configured substantially in the same manner as the first culturing tank 12a, except that the capacity of the accommodation units 18 is smaller than the capacity of the accommodation units 18 of the first culturing tank 12a. The guide units 24 provided in the accommodation units 18 of the second culturing tank 12b, and the guide units 24 provided in the accommodation units 18 of the first culturing tank 12a, are set to have substantially the same inner diameter, in other words, to have substantially the same capacity. For this reason, the ratio of the capacity of the circulation units 26 to the capacity of the guide units 24 is formed to be larger in the first culturing tank 12a than in the second culturing tank 12b.

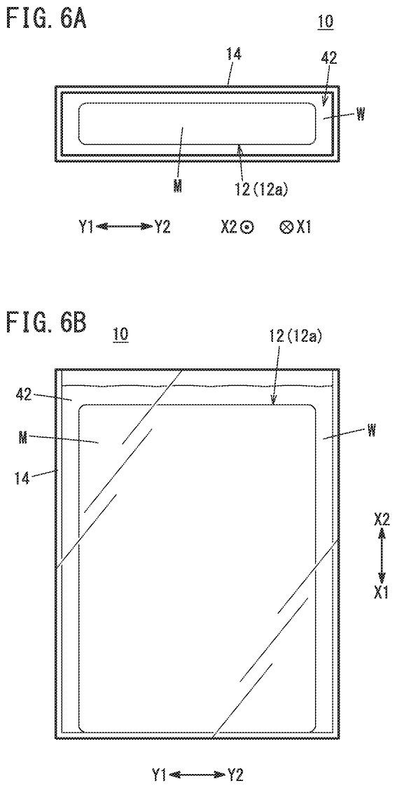

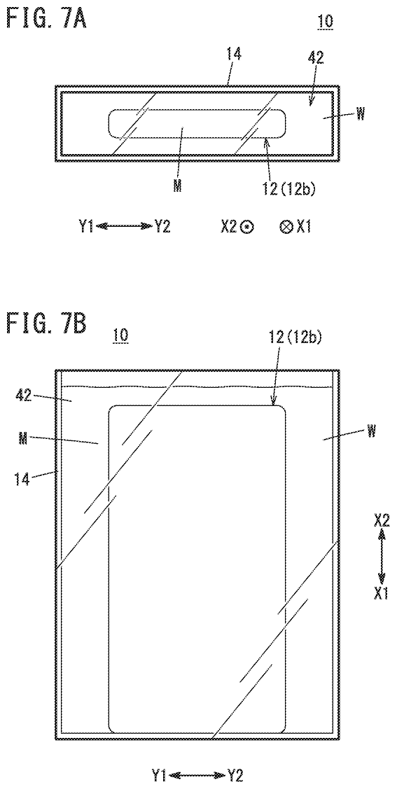

The water storage tank 14 shown in FIG. 5, in the same manner as the main body portion 16, is formed from a material that is flexible and transmits light, such as linear low density polyethylene (LLDPE). Moreover, the water storage tank 14 may be formed from a material that transmits light such as acrylic resin, polycarbonate resin, or glass. The water storage tank 14 includes the water storage unit 42 in which stored water W is stored. The stored water W is a liquid that transmits light, such as water. The water storage unit 42 is set to be larger than the external dimensions of the first culturing tank 12a and the second culturing tank 12b, and as shown in FIG. 6A to 7B, it is possible to install the culturing tank 12, which is selected from among the first culturing tank 12a and the second culturing tank 12b, in the water storage unit 42. Moreover, although the water storage tank 14 in the shape of a casing is illustrated in FIGS. 5 to 7B, the water storage tank 14 may be formed in various shapes in which the water storage unit 42 can be formed, and for example, may be formed in a bag shape.

The culture device 10 is basically configured in the manner described above. A description will be presented concerning a culture method according to the present embodiment in which the culture device 10 is used. In such a culture method, first, as shown in FIG. 5, after or before the stored water W is stored in the water storage unit 42 of the water storage tank 14, a determination step of determining the outdoor environment of the installation location of the culture device 10 is performed. In the determination step, the outdoor environment is determined from the outside air temperature or the sunlight intensity as detected by a temperature sensor, a sunlight sensor, or the like (not shown), or from a calendar.

Next, a volume occupancy rate adjustment step is carried out. In the volume occupancy rate adjustment step, for example in the winter season, in the case that the temperature of the outdoor environment determined in the determination step is less than or equal to a predetermined temperature suitable for culturing the microalgae, from among the first culturing tank 12a and the second culturing tank 12b, the first culturing tank 12a for which the capacity of the accommodation units 18 thereof is larger is selected as the selected culturing tank 12. Then, in a state in which the first culturing tank 12a (the selected culturing tank 12) prior to the contents M being accommodated in the accommodation units 18 is installed in the interior of the water storage unit 42, the contents M are accommodated in the accommodation units 18 from the culture solution supply mechanism through the culture solution supply port 30 (see FIG. 6A and FIG. 6B).

On the other hand, in the volume occupancy rate adjustment step, for example in the summer season, in the case that the temperature of the outdoor environment determined in the determination step is higher than the predetermined temperature suitable for culturing the microalgae, from among the first culturing tank 12a and the second culturing tank 12b, the second culturing tank 12b for which the capacity of the accommodation units 18 thereof is smaller is selected as the selected culturing tank 12. Then, in a state in which the second culturing tank 12b (the selected culturing tank 12) prior to the contents M being accommodated in the accommodation units 18 is installed in the interior of the water storage unit 42, the contents M are accommodated in the accommodation units 18 from the culture solution supply mechanism through the culture solution supply port 30 (see FIG. 7A and FIG. 7B).

More specifically, in the volume occupancy rate adjustment step, the selected culturing tank 12, which is selected from among the first culturing tank 12a and the second culturing tank 12b based on the determination result of the determination step, is arranged inside the water storage unit 42. Consequently, it is possible to adjust the volume occupancy rate, which is a ratio of the content volume of the contents M in the accommodation units 18 disposed in the water storage unit 42, to the total volume of the content volume and the stored water volume of the stored water W that is stored in the water storage unit 42. At this time, it is preferable that, as the outside air temperature increases, the volume occupancy rate is reduced. As one example thereof, there may be cited a case in which the volume occupancy rate in the winter season is set to 0.21, and the volume occupancy rate in the summer season is set to 0.074.

Next, a gas supplying step is performed in which the gas is supplied from the gas supply mechanism to the guide units 24 of the accommodation units 18 via the gas supply ports 28 in the culturing tank 12 disposed in the water storage unit 42. Consequently, since the culture solution flow F can be generated in the accommodation units 18, the gas can be supplied to the entirety of the microalgae while the microalgae are circulated. Further, since the water storage tank 14, the stored water W, and the accommodation units 18, respectively, transmit light, the microalgae inside the accommodation units 18 can be irradiated with light from various directions. As a result, the microalgae proliferate in the accommodation units 18 while performing photosynthesis.

After the microalgae have been sufficiently proliferated in the accommodation units 18 by culturing the microalgae in the manner described above, a collection step is performed in which the contents M are collected from the interior of the accommodation units 18 through the collection port 34. The microalgae are obtained by separating the contents M into the microalgae and the culture solution.

From the above, in the culture device 10 according to the present embodiment, the first culturing tank 12a and the second culturing tank 12b in which the capacities of the accommodation units 18 thereof differ from each other are provided, and the selected culturing tank 12 which is selected from among the first and second culturing tanks can be interchangeably disposed in the water storage unit 42 of the water storage tank 14. As a result, it is possible to adjust the volume occupancy rate, which is a ratio of the content volume in the accommodation units 18 disposed in the water storage unit 42, to the total volume of the stored water volume and the content volume.

Therefore, by reducing the volume occupancy rate and increasing the ratio of the stored water volume to the total volume, for example, even in the case that the outdoor environment is in the summer season or the like and the outside air temperature at the installation location of the culture device 10 is higher than the predetermined temperature, the cooling action of the stored water W is capable of suppressing a rise in temperature inside the accommodation units 18.

Further, by reducing the volume occupancy rate, the content volume becomes smaller, whereby the light irradiation efficiency with respect to the microalgae inside the accommodation units 18 becomes higher. In the case that the outside air temperature at the installation location of the culture device 10 is higher than the predetermined temperature, since the activity of the microalgae is increased, it is possible to promote photosynthesis and increase the culturing efficiency by increasing the light irradiation efficiency.

On the other hand, the culture solution in which the microalgae is contained is colored, such as a green color, and absorbs infrared rays and the temperature thereof rises more easily than the stored water W which is transparent. Therefore, by increasing the volume occupancy rate, and increasing the ratio of the content volume to the total volume, for example, even in the case that the outdoor environment is in the winter season or the like and the outside air temperature at the installation location of the culture device 10 is lower than the predetermined temperature, lowering of the temperature inside the accommodation units 18 can be suppressed.

Further, by increasing the volume occupancy rate, the content volume becomes greater, whereby the light irradiation efficiency with respect to the microalgae inside the accommodation units 18 becomes lower. In the case that the outside air temperature at the installation location of the culture device 10 is lower than the predetermined temperature, since the activity of the microalgae is lowered, the occurrence of light-inhibition can be suppressed by lowering the light irradiation efficiency. In other words, by suppressing the irradiation intensity of the light with respect to the activity of the microalgae from becoming excessive, it is possible to suppress a decrease in the photosynthetic ability. As a result, the microalgae can be cultured satisfactorily even under a low-temperature environment such as in the winter season.

Accordingly, in the culture device 10 and the culture method according to the present embodiment, for example, it becomes easy to maintain the culture solution at a temperature suitable for culturing the microalgae. Further, it also becomes possible to adjust the light irradiation efficiency with respect to the microalgae in the accommodation units 18 in accordance with the outside air temperature at the installation location of the culturing tanks 12. In accordance with these features, the microalgae can be cultured satisfactorily.

In the culture device 10 according to the above-described embodiment, each of the first culturing tank 12a and the second culturing tank 12b (the plurality of culturing tanks 12) is equipped with the main body portion 16 made up from a material that transmits light, the accommodation units 18 are formed on the inner side surrounded by the enclosures 40 formed by joining together the inner wall surfaces of the main body portion 16, the main body portion 16 includes the joint members 22 formed by joining together the inner wall surfaces of the main body portion 16, and extending in the direction in which the gas is supplied, the guide units 24 and the circulation units 26 that are disposed in the interior of the accommodation units 18, that lie adjacent to each other with the joint members 22 being interposed therebetween, and that extend respectively along the direction in which the joint members 22 extend, and the gas supply ports 28 that are capable of supplying the gas to the guide units 24, the gas is supplied to the guide units 24 from the lower side toward the upper side when the main body portion 16 is installed at the installation location, and the guide units 24 and the circulation units 26 communicate with each other respectively via the guide unit inlet ports 44 provided on the upstream side in the direction in which the gas is supplied, and the guide unit outlet ports 46 provided on the downstream side in the direction in which the gas is supplied.

In this case, the culture solution flow F can be generated in the accommodation units 18, by supplying the gas necessary for culturing the microalgae from the gas supply ports 28, and allowing the gas to flow through the guide units 24. Since the microalgae can be circulated due to the culture solution flow F, the gas can be effectively supplied to the entirety of the microalgae inside the accommodation units 18, and photosynthesis can be promoted.

In addition, in the culture device 10, by means of a simple configuration in which the inner wall surfaces of the main body portion 16 are joined together and thereby form the joint members 22, it is possible to provide the guide units 24, the circulation unit 26, and the like for generating the culture solution flow F. Further, for example, there is no need to provide and drive a special configuration such as a water pump in order to generate the culture solution flow F. In accordance with these features, according to the culture device 10, it is possible to culture the microalgae satisfactorily with a simple configuration, while suppressing an increase in energy consumption.

Moreover, the plurality of culturing tanks 12 are not limited to those configured in the manner described above, and various configurations can be adopted therefor in which the capacities of the accommodation units 18 in which the contents M are accommodated differ from each other.

In the first culturing tank 12a and the second culturing tank 12b of the culture device 10 according to the above-described embodiment, the sizes of the main body portions 16 themselves differ from each other. However, the present invention is not particularly limited to this feature. For example, by changing the positions of the joint members 22 relative to the enclosures 40 for each of the first culturing tank 12a and the second culturing tank 12b (the plurality of culturing tanks 12), the capacity of the guide units 24 may be made smaller, and the capacities of the accommodation units 18 may be made to differ relatively without changing the size of the main body portion 16. In this case, since the plurality of culturing tanks 12 can be formed from the same material (the main body portion 16), the configuration of the culture device 10 can be further simplified, and the manufacturing cost can be reduced.

In the first culturing tank 12a and the second culturing tank 12b (the plurality of culturing tanks 12) of the culture device 10 according to the above-described embodiment, as the capacity of the accommodation units 18 is larger, the ratio of the capacity of the circulation units 26 to the capacity of the guide units 24 becomes greater. When the ratio of the capacity of the circulation units 26 to the capacity of the guide units 24 is increased, the circulation speed at which the contents M are circulated within the entirety of the accommodation units 18 due to the culture solution flow F is likely to become slower.

By setting the capacity ratio between the guide units 24 and the circulation units 26 of the plurality of culturing tanks 12 in the manner described above, for example, in the case that the outside air temperature at the installation location of the culture device 10 is low, it becomes possible to install, in the water storage unit 42, the selected culturing tank 12 in which the circulation speed of the contents M inside the accommodation units 18 is slow and the temperature of the contents M is unlikely to be lowered. On the other hand, in the case that the outside air temperature at the installation location of the culture device 10 is high, it becomes possible to install, in the water storage unit 42, the selected culturing tank 12 in which the circulation speed of the contents M inside the accommodation units 18 is fast and the temperature of the contents M is likely to be lowered. With these features, it becomes easier to maintain the culture solution at a temperature suitable for culturing the microalgae.

The ratio of the capacity of the circulation units 26 to the capacity of the guide units 24 in the first culturing tank 12a and the second culturing tank 12b is not limited to the relationship discussed above. Further, the capacities of the guide units 24 may differ from each other in the first culturing tank 12a and the second culturing tank 12b.

In the culture method according to the above-described embodiment, the culture device 10 is equipped with the plurality of culturing tanks 12 in which the capacities of the accommodation units 18 thereof differ from each other, and in the volume occupancy rate adjustment step, the volume occupancy rate is set by disposing, within the water storage unit 42, the culturing tank 12 which is selected from among the plurality of culturing tanks 12. However, the present invention is not particularly limited to this feature. For example, the culture device 10 may be equipped with one culturing tank 12, and a plurality of the water storage tanks 14 in which the capacities of the water storage units 42 thereof differ from each other. Then, in the volume occupancy rate adjustment step, the volume occupancy rate may be set by disposing the culturing tank 12 within the water storage unit 42 of the water storage tank 14 selected from among the plurality of water storage tanks 14.

In the volume occupancy rate adjustment step of the culture method according to the above-described embodiment, as the outside air temperature increases, the volume occupancy rate is reduced. In this case, as described above, the temperature inside the accommodation units 18 can be maintained at a temperature that is suitable for culturing the microalgae, and the light irradiation efficiency with respect to the microalgae inside the accommodation units 18 can be adjusted in accordance with the activity of the microalgae. As a result, it is possible to culture the microalgae satisfactorily.

In the determination step of the culture method according to the above-described embodiment, the volume occupancy rate is set based on the outside air temperature detected by a temperature sensor or the like. However, the volume occupancy rate may be set based on the sunlight intensity as measured using a non-illustrated sunlight sensor or the like. Further, for example, a period from April to November may be set beforehand as the summer season, and a period from December to March may be set beforehand as the winter season, and the volume occupancy rate may be set based on a calendar.

The present invention is not particularly limited to the above-described embodiment, and various modifications are capable of being made thereto without departing from the essence and gist of the present invention.

For example, in the above-described embodiment, the culture device 10 is equipped with the first culturing tank 12a and the second culturing tank 12b as the plurality of culturing tanks 12. Further, in the case that the outside air temperature at the installation location of the culture device 10 is less than or equal to the predetermined temperature, the first culturing tank 12a is selected as being the selected culturing tank 12. In addition, in the case that the outside air temperature at the installation location of the culture device 10 is higher than the predetermined temperature, the second culturing tank 12b is selected as being the selected culturing tank 12.

However, the culture device 10 may include, as the plurality of culturing tanks 12, three or more culturing tanks 12 in which the capacities of the accommodation units 18 thereof differ from each other. In this case, for example, each of the culturing tanks 12 may be assigned a predetermined temperature range (a sunlight intensity range, a range of months in a calendar, or the like) depending on the capacity of the accommodation units 18, and the selected culturing tank 12 may be determined depending on the temperature range (the sunlight intensity range, the range of months in the calendar, or the like) within which the outside air temperature at the installation location of the culture device 10 falls. The predetermined temperature, the predetermined sunlight intensity, the predetermined temperature range, the predetermined sunlight intensity range, and the range of months in the calendar in order to determine the selected culturing tank 12 can be set appropriately in accordance with the type of the microalgae to be cultured.

### REFERENCE SIGNS LIST

10 . . . culture device
12 . . . culturing tank
14 . . . water storage tank
16 . . . main body portion
18 . . . accommodation unit
22 . . . joint member
24 . . . guide unit
26 . . . circulation unit
42 . . . water storage unit
44 . . . guide unit inlet port
46 . . . guide unit outlet port
M . . . contents
W . . . stored water

1. What is claim is:

1. A culture device that cultures microalgae in a culture solution, the culture device comprising:
a plurality of culturing tanks each including an accommodation unit that transmits light and is configured to accommodate the culture solution and the microalgae, wherein capacities of the accommodation units differ from each other; and
a water storage tank including a water storage unit which transmits light and in which stored water is stored,
wherein the culturing tank selected from among the plurality of culturing tanks is interchangeably disposed inside the water storage unit.

2. The culture device according to claim 1, wherein
each of the plurality of culturing tanks is equipped with a main body portion made up from a material that transmits light,
the accommodation unit is formed on an inner side surrounded by an enclosure formed by joining together inner wall surfaces of the main body portion, and a gas is supplied to the accommodation unit,
the main body portion includes:
a joint member formed by joining together the inner wall surfaces of the main body portion, and extending in a direction in which the gas is supplied;
a guide unit and a circulation unit that are disposed in an interior of the accommodation unit, are adjacent to each other with the joint member being interposed therebetween, and lie respectively along a direction in which the joint member extends; and
a gas supply port configured to enable the gas to be supplied to the guide unit,
the gas is supplied to the guide unit from a lower side toward an upper side when the main body portion is installed at an installation location, and
the guide unit and the circulation unit communicate with each other via a guide unit inlet port provided on an upstream side in the direction in which the gas is supplied, and a guide unit outlet port provided on a downstream side in the direction in which the gas is supplied.

3. The culture device according to claim 2, wherein
the capacity of the accommodation unit differs for each of the plurality of culturing tanks by a change in a position of the joint member relative to the main body portion.

4. The culture device according to claim 2, wherein
in the plurality of culturing tanks, as the capacity of the accommodation unit is larger, a ratio of a capacity of the circulation unit to a capacity of the guide unit becomes greater.

5. A culture method using a culture device 0-0+that cultures microalgae in a culture solution,
wherein the culture device includes:
a culturing tank including an accommodation unit that transmits light and is configured to accommodate the culture solution and the microalgae as contents; and
a water storage tank including a water storage unit which transmits light and in which stored water is stored, the culturing tank being allowed to be installed inside the water storage unit,
the culture method comprising:
a determination step of determining an outdoor environment of an installation location in which the culture device is installed; and
a volume occupancy rate adjustment step of setting, based on a determination result of the determination step, a volume occupancy rate which is a ratio of a content volume of the contents accommodated in the accommodation unit inside the water storage unit, to a total volume of the content volume and a stored water volume of the stored water.

6. The culture method according to claim 5, wherein
the culture device includes a plurality of the culturing tanks in which capacities of the accommodation units thereof differ from each other, and
in the volume occupancy rate adjustment step, the volume occupancy rate is set by disposing the culturing tank selected from among the plurality of culturing tanks, inside the water storage unit.

7. The culture method according to claim 5, wherein, in the volume occupancy rate adjustment step, the volume occupancy rate is reduced as the outside air temperature increases.
